(19) United States
(12) Patent Application Publication
(10) Pub. No.: US 2009/0062735 A1
(43) Pub. Date: Mar. 5, 2009

(54) INTRODUCER FOR CANNULA AND METHOD

(76) Inventors: Robert H. Bartlett, Ann Arbor, MI (US); Kenneth R. Jonkman, Marne, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/145,763

(22) Filed: Jun. 25, 2008

Related U.S. Application Data
(60) Provisional application No. 60/968,161, filed on Aug. 27, 2007.

(51) Int. Cl. A61M 5/32; A61M 29/00

(52) U.S. Cl. 604/104; 604/164.11

(57) ABSTRACT

An introducer for a blood vessel cannula has a main elongate portion adapted for insertion into a blood vessel cannula. A first end of a transition portion is adjacent to and extends from the main elongate portion. The first end of the transition portion has an outside diameter larger than an outside diameter of a second end of the transition portion. A first end of an extended portion is adjacent to and extends from the second end of the transition portion and the first end of the extended portion has an outside diameter that is larger than an outside diameter of a second end of the extended portion. A distal end portion is adjacent the second end of the extended portion and has an aperture. A lumen extends through the aperture, main elongate portion, the transition portion, the extended portion, and the distal end portion.

### CROSS REFERENCE TO RELATED APPLICATION

This application claims benefit under 35 U.S. C. §119(e) of provisional application Ser. No. 60/968,161, filed Aug. 27, 2007, entitled INTRODUCER FOR CANNULA AND METHOD. This application is also related to application Ser. No. ______, entitled CANNULA REINFORCING BAND AND METHOD, filed on ______, and application Ser. No. ______, entitled COAXIAL VENAL CANNULA AND METHOD, filed on ______. The entire contents of each of the aforementioned applications are incorporated herein by reference.

### BACKGROUND OF THE PRESENT INVENTION

The present invention relates to an introducer and the like, and in particular to an introducer for use with a cannula and a method related to the same.

### SUMMARY OF THE INVENTION

One aspect of the present invention is an introducer for a blood vessel cannula having a main elongate portion made from a flexible material and having an outside diameter adapted for insertion into a blood vessel cannula. A transition portion has a first end and a second end. The first end of the transition portion is adjacent to and extends from the main elongate portion wherein the first end of the transition portion has an outside diameter larger than an outside diameter of the second end of the transition portion. An extended portion has a first end and a second end. The first end of the extended portion is adjacent to and extends from the second end of the transition portion and the first end of the extended portion has an outside diameter that is larger than an outside diameter of the second end of the extended portion. An extended portion has a first end and a second end. The first end of the extended portion is adjacent to and extends from the second end of the transition portion and the first end of the extended portion has an outside diameter that is larger than an outside diameter of the second end of the extended portion. A distal end portion is adjacent the second end of the extended portion and has an aperture extending therethrough. A lumen extends from the aperture through the main elongate portion, the transition portion, the extended portion, and the distal end portion.

Another aspect of the present invention is a cannula insertion system having a cannula having a proximate end, a distal end and a cannula lumen extending therethrough. An introducer extends through the cannula lumen. The introducer includes a main elongate portion made from a polymeric material and has an outside diameter adapted for insertion into the cannula. A transition portion has a first end and a second end. The first end is adjacent to and extends from the main elongate portion and has an outside diameter larger than an outside diameter of the second end. An extended portion extends from the second end of the transition portion and has an outside diameter less than an outside diameter of the main elongate portion and has greater flexibility than the main elongate portion. A distal end portion is adjacent the extended portion and has an aperture extending therethrough. An introducer lumen extends through the main elongate portion, the transition portion, the extended portion, and the distal end portion.

Another aspect of the present invention is a method of introducing a cannula into the body of a patient that includes providing a cannula having an inner lumen extending therethrough. An introducer is provided that has an elongate shape and is adapted for insertion into the cannula. A main elongate portion is formed on the introducer. An elongate distal portion is formed on the introducer that is smaller in outside diameter than the main elongate portion, and that is more flexible than the main elongate portion. A transition portion is formed on the introducer between the main elongate portion and the distal portion and has a generally frusto-conical shape. A cavity is formed in the introducer that extends through the main elongate portion, the transition portion and the distal portion. The introducer is inserted into the lumen of the cannula. The introducer and cannula are inserted into the body of a patient and positioned inside the body of the patient. The introducer is then removed from the lumen of the cannula.

Yet another aspect of the present invention is a dilator for dilating a blood vessel having a main elongate portion made from a flexible material and having an outside diameter adapted for insertion into a blood vessel cannula. A transition portion has a first end and a second end. The first end of the transition portion is adjacent to and extends from the main elongate portion wherein the first end of the transition portion has an outside diameter larger than an outside diameter of the second end of the transition portion. An extended portion has a first end and a second end. The first end of the extended portion is adjacent to and extends from the second end of the transition portion and the first end of the extended portion has an outside diameter that is larger than an outside diameter of the second end of the extended portion. An extended portion has a first end and a second end. The first end of the extended portion is adjacent to and extends from the second end of the transition portion and the first end of the extended portion has an outside diameter that is larger than an outside diameter of the second end of the extended portion.

These and other features, advantages and objects of the present invention will be further understood and appreciated by those skilled in the art upon studying the following specification, claims, and appended drawings.

### BRIEF DESCRIPTION OF THE DRAWINGS

FIG. 1 is an enlarged partial perspective view of an introducer and cannula of one embodiment of the present invention;

FIG. 2 is a partial perspective view of the introducer and cannula assembly of FIG. 1;

FIG. 3 is a top elevational view of the introducer of FIG. 1 inside another embodiment of a cannula;

FIG. 4 is a top elevational view of an introducer with a guide wire extending therethrough;

FIG. 5 is an enlarged side cross-sectional view of the introducer of FIG. 4 with the guide wire removed;

FIG. 6 is an enlarged side cross-sectional elevational view of FIG. 5 taken at line VI;

FIG. 7 is a bottom perspective view of a distal end of a small introducer;

FIG. 8 is a bottom perspective view of the distal end of a large introducer;

FIG. 9 is a side elevational view of the distal end of a dilator with a guide wire extending through an aperture at the distal end of the dilator;

FIG. 10 is a top elevational view of an introducer and guide wire in a curved orientation;

FIG. 11 is a kit for use in placing a cannula in the body of a patient; and

FIG. 12 is a set of dilators for use in dilating the blood vessels of a patient.

### DETAILED DESCRIPTION OF PREFERRED EMBODIMENTS

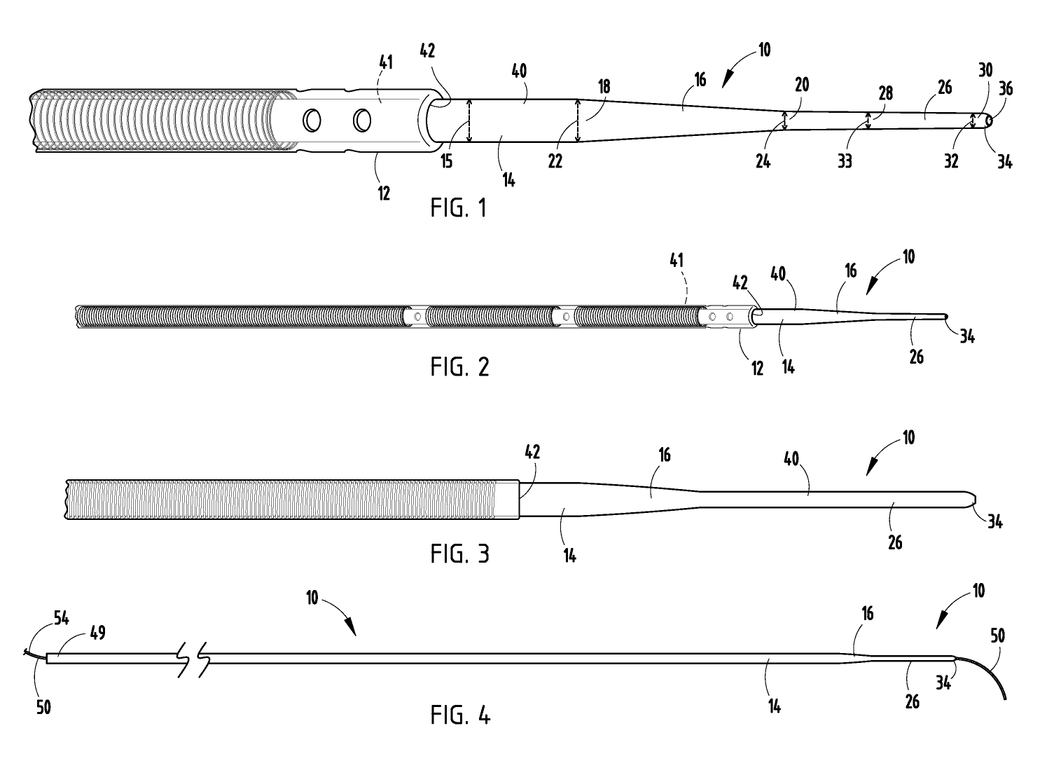

For purposes of description herein the terms “upper”, “lower”, “right”, “left”, “rear”, “front”, “vertical”, “horizontal” and derivatives thereof shall relate to the invention as oriented in FIG. 1. However, it is to be understood that the invention may assume various alternative orientations and step sequences, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

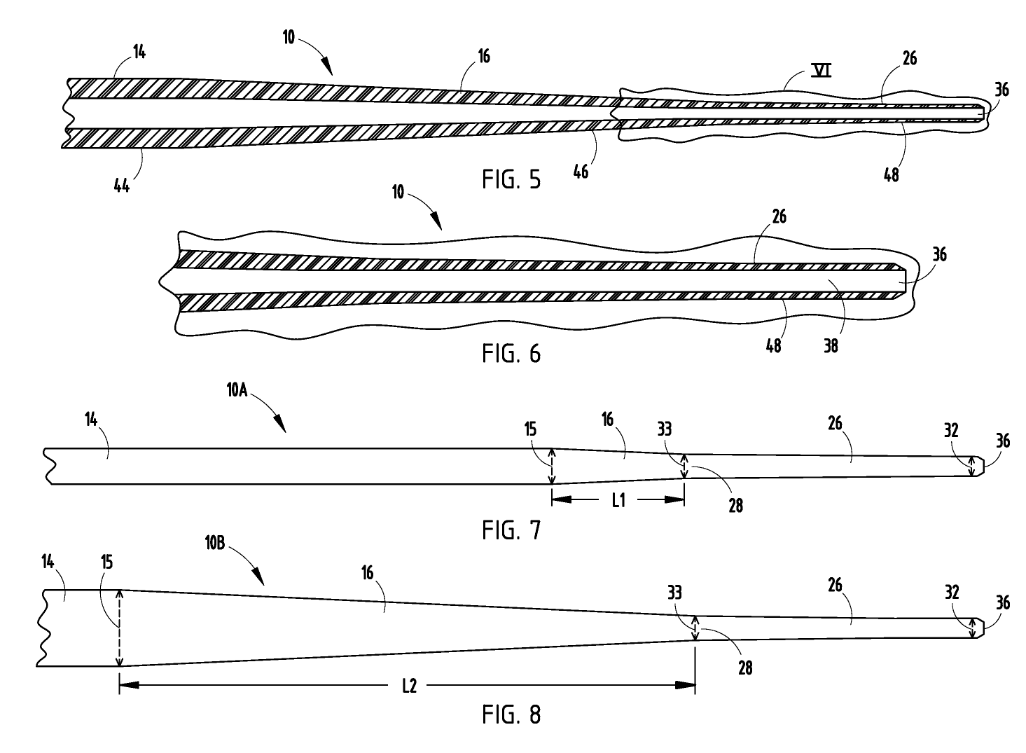

As illustrated in FIG. 1, the reference numeral 10 generally designates an introducer for a blood vessel cannula 12 having a main elongate portion 14 made from a flexible material and which has an outside diameter 15 adapted for insertion into a blood vessel cannula. A transition portion 16 has a first end 18 and a second end 20. The first end 18 of the transition portion 16 is adjacent to and extends from the main elongate portion 14 and has an outside diameter 22 that is larger than an outside diameter 24 of the second end 20 of the transition portion 16. An extended portion 26 has a first end 28 and a second end 30. The first end 28 of the extended portion 26 is adjacent to and extends from the second end 20 of the transition portion 16. The second end 30 of the extended portion 26 has an outside diameter 32 that is smaller than an outside diameter 33 of the first end 28 of the extended portion 26. A distal end portion 34 is adjacent the second end 30 of the extended portion 26 and has an aperture 36 extending therethrough. A lumen 38 (FIGS. 5 and 6) extends from the aperture 36 through the distal end portion 34, the extended portion 26, the transition portion 16, and the main elongate portion 14.

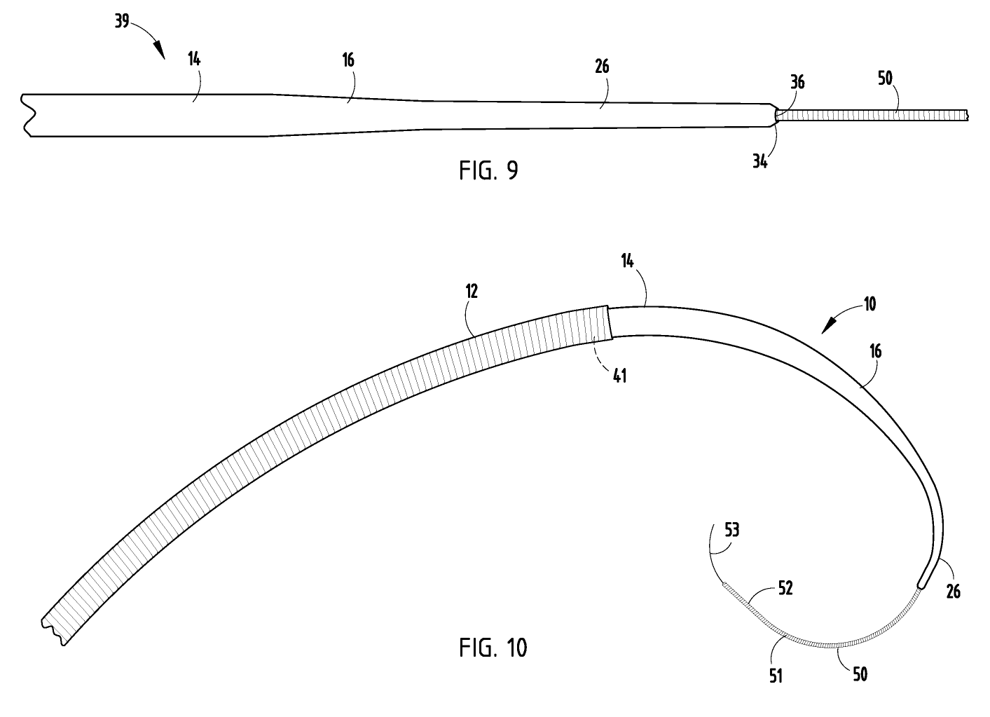

The introducer 10 may also function as a dilator 39 (FIG. 9) of blood vessels. The introducer 10 is used to facilitate placement of a cannula 12 inside the body of a patient at a predetermined location. The dilator 39 is used to dilate blood vessels along the path of the guide wire 50. It should be understood that any and all structural features detailed with respect to the introducer 10 may also apply to the dilator 39.

Referring now to FIGS. 1-3, the introducer 10 includes a smooth external surface 40 that allows it to be easily slid inside a lumen 41 of the cannula 12. The introducer 10 may be made from moldable or heat formable plastic, silicone or other formable materials. In use, the transition portion 16, the extended portion 26, and the distal end portion 34 may extend from a distal end 42 of the cannula 12. However, a part of the main elongate portion 14 may also extend from the distal end 42 of the cannula 12, as shown in FIG. 1.

Referring now to FIGS. 4-6, the introducer 10 may be very long or relatively short, as compared to the length of a given cannula 12 but will usually be longer than the cannula 12. The main elongate portion 14 may vary in length, but generally has a fixed diameter depending on the application. The main elongate portion 14 will generally have a larger diameter when used with a larger cannula 12. Large cannulas 12 are often used in large blood vessels such as the aorta or superior vena cava. Alternatively, the main elongate portion 14 will have a smaller diameter when used with a smaller cannula 12. Smaller cannulas 12 are often necessary in smaller adult blood vessels or in the blood vessels of children. The main elongate portion 14, transition portion 16, and extended portion 26 include walls 44, 46, 48, respectively, that define the introducer lumen 38. Generally, the wall 48 of the extended portion 26 will not be as thick as the wall 48 of the main elongate portion 14. The thickness of the wall 48 of the extended portion 26 will generally range from 0.006 to 0.015 inches thick and will generally be approximately 0.010 inches. However, it is contemplated that the thickness of the wall 48 could be greater than 0.015 inches or less than 0.006 inches.

The main elongate portion 14 is adjacent to the transition portion 16 and is generally integral therewith. However, it should be understood that the transition portion 16 could be made from a different material than the main elongate portion 14 and be a separate piece that is attached to the main elongate portion 14. The transition portion 16 has a generally frusto-conical shape with the first end 18 of the transition portion 16 being connected with the main elongate portion 14 and the second end 20 being connected to the extended portion 26. The transition portion 16 generally tapers outwardly from the second end 20 to the first end 18. The transition portion 16 may taper outwardly at approximately a 2.5 degree angle.

In addition, the extended portion 26 may have a generally frusto-conical shape or a generally cylindrical shape. The first end 28 of the extended portion 26 is adjacent to and connected with the second end 20 of the transition portion 16. The extended portion 26 is generally, approximately, one inch long (25.4 mm), although the general range of the length of the extended portion 26 is 0.5 inches (12.7 mm) to 1.5 inches (38.1 mm). It is contemplated that the extended portion 26 could be considerably longer than 1.5 inches or shorter than 0.5 inches. In addition, the extended portion 26 tapers outwardly from the second end 30 to the first end 28 at approximately a 0.7 degree angle. The degree of taper could be as little as zero degrees or greater than 1.0 degree. When the degree of taper is zero degrees, the outside diameter of the first and second ends will be approximately equal.

As shown in FIGS. 5 and 6 and briefly discussed above, the aperture 36 extends through the distal end portion 34. The distal end portion 34 tapers downwardly toward the aperture 36 at approximately a thirty degree angle, although it is contemplated that the angle could be more or less than thirty degrees. The aperture 36 is generally aligned with the lumen 38 that extends through the distal end portion 34, the extended portion 26, the transition portion 16 and the main elongate portion 14. The aperture 36 is adapted to receive a guide wire 50 prior to the introducer 10 being inserted into the body of the patient.

Referring now to FIGS. 7 and 8, the transition portion 16 has a length L that may vary depending on the size of the main elongate portion 14 and extended portion 26. More specifically, the length of the transition portion 16 may vary depending on the outside diameter 33 of the first end 28 of the extended portion 26 and the outside diameter 15 of the main elongate portion 14. The smaller introducer 10A shown in FIG. 7 illustrates that when the difference in size between the outside diameter 15 of the main elongate portion 14 and outside diameter 33 of the first end 28 of the extended portion 26 is small, the transition portion 16 will have a length L1 that is smaller. The larger introducer 10B illustrated in FIG. 8 shows that as the difference in diameter size increases between the outside diameter 15 of the main elongate portion 14 and the outside diameter 33 of the first end 28 of the extended portion 26, the transition portion 16 will have a length L2 that is generally longer. Although the transition portion 16 will often taper outwardly from the second end 20 to the first end 18 at approximately 2.5 degrees, it should be understood that other angles of taper may also be used that will affect the length of the transition portion 16.

Referring now to FIGS. 9 and 10, the guide wire 50 is designed to slide into the introducer 10 at a distal end 49 (FIG. 4), pass through the lumen 38 of the introducer 10, and extend out of the distal end portion 34. As shown in FIG. 10, the transition portion 16 and extended portion 26 provide substantial flexibility to the introducer 10 such that navigating through the complicated vascular system of a patient can be done with minimal likelihood of kinking the guide wire 50 or damaging blood vessels with the introducer 10 and/or cannula 12. FIG. 10 shows the transition portion 16 and extended portion 26 flexing to over a 90 degree angle, easily following the path provided by the guide wire 50. The guide wire generally includes a coil portion 51 that wraps around a guide strand 53.

Generally, during a medical procedure, a physician feeds the guide wire 50 into a patient until a distal end 52 of the guide wire 50 has reached a desired location. Once the guide wire 50 has been properly placed, the physician may need to dilate the blood vessels through which the guide wire 50 extends. In this instance, a proximal end 54 (FIG. 4) of the guide wire 50 is fed into the aperture 36 of the dilator 39. The dilator 39 is then inserted into the body along the guide wire 50 and provides gradual, controlled force dilation of the blood vessels along the path of the guide wire 50 extending into the patient until it reaches a specific location predetermined by the physician. The extended portion 26 of the dilator 39 is adapted to bend and flex through the complex lattice of blood vessels in the patient following the guide wire 50 and acclimating the blood vessels along the path of the guide wire 50 to the size of the main elongate portion 14 of the dilator 39. After the dilator 39 has been allowed to slightly enlarge or stretch the blood vessels, the dilator 39 is withdrawn from the body of the patient.

The physician then inserts a larger introducer 10 into a preselected cannula 12 having a proximal end (not shown) and a distal end 42. The introducer 10 is inserted into the proximal end of the cannula 12 until a desired amount of the main elongate portion 14, the transition portion 16, and the extended portion 26 extend beyond the distal end 42 of the cannula 12. The proximal end 54 of the guide wire 50 is then fed into the introducer 10 and the introducer 10, as well as the cannula 12, are fed into the body of the patient along the extent of the guide wire 50. Once the cannula 12 has reached the desired position, the physician withdraws the guide wire 50 through the lumen 38 of the introducer 10 and out of the body of the patient. The introducer 10 is then withdrawn from the lumen 41 of the cannula 12. Alternatively, the physician may withdraw the introducer 10 and guide wire 50 simultaneously. It should be understood that the dilation or cannulization procedures discussed above may be conducted together or separately.

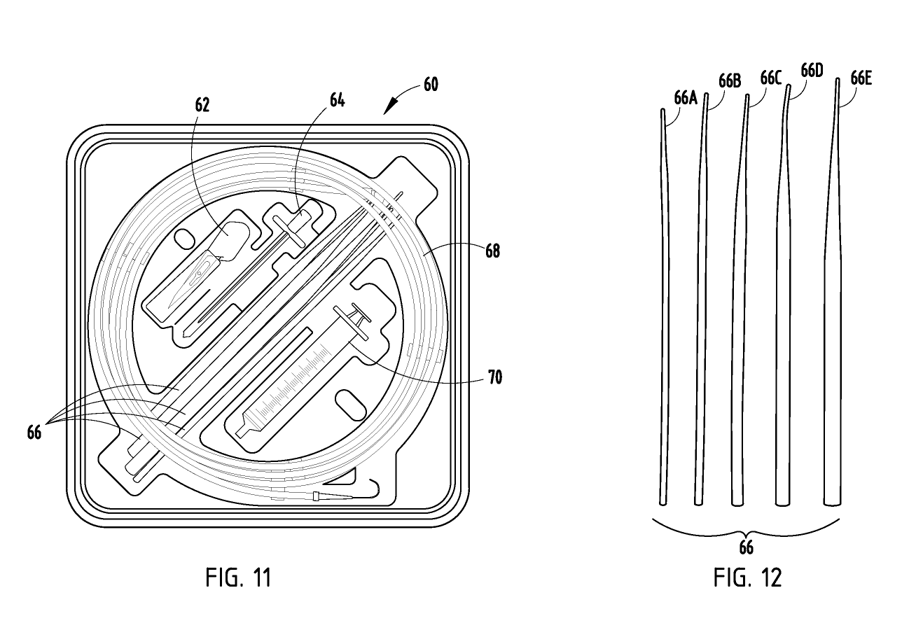

Referring now to FIG. 11, a cannula kit 60 includes a scalpel or other sharp cutting instrument 62 designed for penetrating the epidermis of a patient during a cannulization procedure. The kit also includes a percutaneous needle 64, a plurality of dilators 66 of varying diameters, a sheathed guide wire 68, and a syringe 70. Each of these items is designed for use in a cannulization procedure. The dilators 66A-66E include varying diameters with the diameter of dilator 66E being the largest and the diameter of dilator 66A being the smallest (FIG. 12). In use, after the percutaneous needle 64 has been inserted through the epidermis of a patient by a physician, the physician can then prepare the opening created by the needle 64 for an introducer by using the dilators 66A-66E in order of smallest to largest. This causes the opening in the epidermis to grow, such that the opening can accommodate the diameter of a large introducer thereby minimizing any tearing that could possibly occur if the introducer was introduced into the opening prior to preparation by one or more dilators 66. It is to be understood that the dilators 66A-66E include the same construction as outlined above with respect to dilator 39 and introducer 10.

The above description is considered that of the preferred embodiments only. Modifications of the invention will occur to those skilled in the art and to those who make or use the invention. Therefore, it is understood that the embodiments shown in the drawings and described above is merely for illustrative purposes and not intended to limit the scope of the invention, which is defined by the following claims as interpreted according to the principles of patent law, including the Doctrine of Equivalents.

The invention claimed is:

1. An introducer for a blood vessel cannula comprising:
a main elongate portion made from a flexible material and having an outside diameter adapted for insertion into a blood vessel cannula;
a transition portion having a first end and a second end, said first end of said transition portion being adjacent to and extending from said main elongate portion wherein said first end of said transition portion has an outside diameter larger than an outside diameter of said second end of said transition portion;
an extended portion having a first end and a second end, said first end of said extended portion being adjacent to and extending from said second end of said transition portion and said first end of said extended portion having an outside diameter that is larger than an outside diameter of said second end of said extended portion;
a distal end portion adjacent said second end of said extended portion and having an aperture extending therethrough; and
a lumen extending from said aperture through said main elongate portion, said transition portion, said extended portion, and said distal end portion.

2. The introducer of claim 1, wherein:
the longitudinal extent of said extended portion is longer than the longitudinal extent of said transition portion.

3. The introducer of claim 1, wherein:
the longitudinal extent of said transition portion is longer than the longitudinal extent of said extended portion.

4. The introducer of claim 1, wherein:
said distal end portion tapers downwardly toward said aperture.

5. The introducer of claim 1, wherein:
said distal end portion tapers downwardly toward said aperture at approximately a thirty degree angle.

6. The introducer of claim 1, wherein:
said extended portion tapers outwardly from said second end to said first end at an angle between approximately zero and one degrees.

7. The introducer of claim 1, wherein:
said extended portion is between 0.5 inches (12.7 mm) and 1.5 inches (38.1 mm) long.

8. The introducer of claim 1, further comprising:
a guide wire extending through said aperture.

9. A cannula insertion system comprising:
a cannula having a proximate end, a distal end and a cannula lumen extending therethrough;
an introducer extending through said cannula lumen, said introducer comprising:
a main elongate portion made from a polymeric material and having an outside diameter adapted for insertion into said cannula;
a transition portion having a first end and a second end, said first end being adjacent to and extending from said main elongate portion and having an outside diameter larger than an outside diameter of said second end;
an extended portion extending from said second end of said transition portion and having an outside diameter less than the outside diameter of said main elongate portion and having greater flexibility than said main elongate portion;
a distal end portion adjacent said extended portion and having an aperture extending therethrough; and
an introducer lumen extending through said main elongate portion, said transition portion, said extended portion, and said distal end portion.

10. The cannula insertion system of claim 9, wherein:
the outside diameter of said second end of said extended portion is larger than an outside diameter of said first end.

11. The cannula insertion system of claim 9, wherein:
the outside diameter of said second end of said extended portion is substantially equal to an outside diameter of said first end.

12. The cannula insertion system of claim 9, wherein:
the longitudinal extent of said extended portion is longer than the longitudinal extent of said transition portion.

13. The cannula insertion system of claim 9, wherein:
the longitudinal extent of said transition portion is longer than the longitudinal extent of said extended portion.

14. The cannula insertion system of claim 9, further comprising:
a guide wire extending through said introducer lumen.

15. A method of introducing a cannula into the body of a patient comprising:
providing a cannula having an inner lumen extending therethrough;
providing an introducer having an elongate shape and adapted for insertion into said cannula;
forming a main elongate portion on said introducer;
forming an elongate distal portion on said introducer that is smaller in outside diameter than said main elongate portion, and that is more flexible than said main elongate portion;
forming a transition portion on said introducer between said main elongate portion and said distal portion that has a generally frusto-conical shape;
forming a cavity in said introducer that extends through said main elongate portion, said transition portion and said distal portion;
inserting said introducer into said lumen of said cannula;
inserting said introducer and cannula into the body of a patient;
positioning said introducer and said cannula inside the body of the patient; and
removing said introducer from said lumen of said cannula.

16. The method of claim 15, further comprising:
forming the elongate distal portion into a generally frusto-conical shape.

17. A dilator for dilating a blood vessel, comprising:
a main elongate portion made from a flexible material and having an outside diameter adapted for insertion into a blood vessel cannula;
a transition portion having a first end and a second end, said first end of said transition portion being adjacent to and extending from said main elongate portion wherein said first end of said transition portion has an outside diameter larger than an outside diameter of said second end of said transition portion; and
an extended portion having a first end and a second end, said first end of said extended portion being adjacent to and extending from said second end of said transition portion and said first end of said extended portion having an outside diameter that is larger than an outside diameter of said second end of said extended portion.

18. The dilator of claim 17, further comprising:
a distal end portion adjacent said second end of said extended portion and having an aperture extending therethrough.

19. The dilator of claim 18, wherein:
the longitudinal extent of said transition portion is longer than the longitudinal extent of said extended portion.

20. The dilator of claim 19, wherein:
said extended portion tapers outwardly from said second end to said first end at an angle between approximately zero and one degrees.
